(19) United States
(12) Patent Application Publication
(10) Pub. No.: US 2023/0255671 A1
(43) Pub. Date: Aug. 17, 2023

(54) 5TH METATARSAL COMPRESSION FIXATION SYSTEM

(71) Applicant: THE GENERAL HOSPITAL CORPORATION, Boston, MA (US)

(72) Inventors: Christopher W. Digiovanni, Milton, MA (US); Daniel Guss, Brookline, MA (US); Soheil Ashkni Esfahani, Malden, MA (US); Gregory Waryasz, Milton, MA (US); Bart Lubberts, Cambridge, MA (US)

(21) Appl. No.: 18/015,019

(22) PCT Filed: Jul. 7, 2021
(86) PCT No.: PCT/US2021/040759

§ 371 Date: Jan. 6, 2023

Related U.S. Application Data
(60) Provisional application No. 63/048,788, filed on Jul. 7, 2020.

(51) Int. Cl. A61B 17/86; A61B 17/72; A61B 17/17

(52) U.S. Cl. CPC A61B 17/8695; A61B 17/7291; A61B 17/1728

(57) ABSTRACT

Systems and devices for compressing a 5^{th} metatarsal fracture of the foot are provided. A bone fixation device is provided that includes an opening for receiving a bone screw. The device also includes at least one hook sized and configured to be placed at the base of the 5^{th} metatarsal bone of the foot. Drill guides and bone tap guides are also provided that include at least one hook sized and configured to placed at the base of the 5^{th} metatarsal bone of the foot.

### CROSS-REFERENCE TO RELATED APPLICATIONS

The present application claims priority to U.S. Provisional Application No. 63,048,788 filed on Jul. 7, 2020 and which is incorporated by reference in its entirety.

### TECHNICAL FIELD

The present application relates to compression fixation devices to surgically repair a 5^{th} metatarsal fracture.

### BACKGROUND

The bones of a normal foot include the calcaneus, talus, navicular, three cuneiforms, cuboid, five metatarsals, and fourteen phalanges. These 26 bones of the foot are divided into the tarsus, metatarsus, and phalanges. The seven tarsal bones are short bones that connect the metatarsals to the leg. The five metatarsals are weight-bearing bones and provide a foundation for the digits. The phalanges provide the skeletal structure for the digits. Functionally, the bones of the foot can also be grouped into the rearfoot (which includes of the calcaneus and talus), the midfoot (which includes the navicular, cuboid, and the three cuneiforms), and the forefoot (which includes the metatarsals and their respective phalanges). The foot bones can also be grouped into a medial or lateral column. The medial column includes the talus, navicular, the three cuneiforms, the first to third metatarsals, and the respective phalanges. The lateral column includes the calcaneus, cuboid, the fourth and fifth metatarsals, and the phalanges.

There are a total of five metatarsals, which are numbered from I to V in a medial to lateral fashion.

With reference to FIG. 3, each metatarsal is a long bone with a base, 12, shaft 14, and head 16. The metatarsals extend anteriorly from the foot and are nearly parallel to each other)(0°-8°. With reference to FIG. 1, because the fifth metatarsal 18 does not protrude as far as any of the other metatarsals, in the articulated skeleton, it is considered the shortest. The base of a metatarsal is usually wedge shaped, presenting with five surfaces. The superior and inferior surfaces are rough for the attachment of ligaments. The medial and lateral surfaces have both flat articular and nonarticular areas. The articular facets are for either the tarsus or metatarsus. The posterior surface is slightly concave and is covered with an articular facet for articulation with the tarsus. The shaft of each metatarsal, when viewed along its dorsal surface, extends directly anteriorly from the base and tapers distal. With reference to FIGS. 1 and 2, the fifth metatarsal 18 shaft differs slightly from the others in that the shaft has a small degree of lateral bowing. When viewed from the side the bone is curved, being convex dorsal and concave plantar. The shaft of each metatarsal varies in thickness but each is generally pyramidal in section. Further, within each metatarsal there is a degree of torsion between the base and the head of the bone. The amount and direction of the torsion varies among the five metatarsals. The head of a metatarsal presents with a smooth convex facet covered with hyaline cartilage that extends farther on the plantar surface than the dorsal surface. The plantar surface is characterized by two condyles separated by a small shallow groove for the passage of the flexor tendons. The lateral plantar condyle is larger than the medial plantar condyle. Both condyles are continuous with the articular surface of the head. Dorsally, a depression known as the surgical neck is present immediately posterior to the articular surface. Posterior to the surgical neck are a medial and lateral tubercle for the attachment of metatarsophalangeal collateral ligaments. Finally, posterior to these tubercles is another depression known as the anatomic neck that represents the point where the primary and secondary centers of ossification have fused.

The fifth metatarsal articulates with three bones: the base of the proximal phalanx, the cuboid, and the fourth metatarsal. The base is generally pyramidal to triangular in shape with the apex pointing inferolateral. The posterior surface is generally triangular in shape and is covered with a single articular facet. The apex generally points laterally and articulates with the cuboid. A distinguishing feature of this metatarsal is a large tuberosity found on the lateral surface. The tuberosity is subcutaneous and rough for the attachment of ligaments and muscles. The medial surface presents with a generally oval facet for the articulation with the fourth metatarsal base. The remaining surfaces are rough for the attachment of ligaments and muscles.

Referring back to FIG. 1, a Jones fractures 13 or 5^{th} metatarsal fracture is a fracture between the base and middle part of the 5^{th} metatarsal bone 18 of the foot. The fracture has a non-union rate that is reportedly as high as 50 percent. This can be caused by 1) poor blood supply in the fracture area within the fifth metatarsal bone and 2) by diastasis and motion of the fracture fragment due to the applied pulling forces of the plantar fascia and peroneal brevis tendon. Patients with a Jones fracture frequently undergo operative management with intramedullary screw fixation which is inserted through the bone canal 20, schematically depicted in FIG. 4. Screw insertion through the base of the fifth metatarsal potentially compromises attachment points of the plantar fascia and peroneal brevis tendon, and appropriate screw length and diameter, as well as significant fracture compression is important for optimal fracture healing.

Aiming to reduce the risk of non-union and faster recovery, surgical treatment using intramedullary screw fixation should maximize fracture compression; keep the articular cartilage intact while optimizing the length of the screw inserted into the intramedullary canal; and minimize destruction of soft tissue insertions during screw placement. The screw should be placed substantially parallel to the cuboid and collinear with the intramedullary cortex.

### SUMMARY

In an aspect, a bone fixation device is provided comprising a top surface, a bottom surface and an opening extending therebetween. The opening is sized and configured to receive a bone screw. The device also comprises at least one hook extending from a back surface of the fixation device. The fixation device is sized and configured to compress a proximal 5^{th} metatarsal fracture.

In another aspect, a drill guide is provided comprising a shaft defining a bore. The bore is sized and configured to receive a K-wire. A handle extends proximally from the shaft and at least one hook extends distally from the shaft. The at least one hook is sized and configured to fit onto the base of a 5^{th} metatarsal bone of the foot. A bone tap guide is also provided that includes a fixation device as described herein as well as a bore sized to receive a bone tap.

### BRIEF DESCRIPTION OF DRAWINGS

FIG. 1 is a schematic illustration of the dorsal surface of a human foot.

FIG. 2 is a schematic partial side view of a human foot.

FIG. 3 is a side view of a metatarsal bone of a human foot.

FIG. 4 is a schematic illustration of a bone canal of a metatarsal bone of a human foot.

FIG. 5 is a perspective view of a bone fixation device according to an aspect of the present disclosure.

FIG. 6 is a side view of a bone fixation device according to an aspect of the present disclosure.

FIG. 7 is a top view of a bone fixation device according to an aspect of the present disclosure.

FIG. 8 is a bottom view of a bone fixation device according to an aspect of the present disclosure.

FIG. 9A-9C are top views of bone fixation devices with different widths according to aspects of the present disclosure.

FIG. 10A-10C are top views of bone fixation devices with different lengths according to aspects of the present disclosure.

FIG. 11A is a bottom view of bone fixation device according to an aspect of the present disclosure.

FIG. 11B is a top view of the bone fixation device of FIG. 11A.

FIGS. 12 and 13 are perspective views of a bone fixation device placed on a fifth metatarsal bone according to an aspect of the present disclosure.

FIG. 14 is a side view of a bone fixation device placed on a fifth metatarsal bone according to an aspect of the present disclosure.

FIG. 15 is a schematic illustration of a screw inserted into a fifth metatarsal bone for treating a Jones fracture.

FIG. 16 a schematic illustration of a bone fixation device through which the screw of FIG. 17 is inserted according to an aspect of the present disclosure.

FIG. 17 is a schematic illustration of a drill guide placed on a fifth metatarsal bone according to an aspect of the present disclosure.

FIG. 18 is a side view of a drill guide according to an aspect of the present disclosure.

FIG. 19 is a side view of a bone tap guide according to an aspect of the present disclosure.

FIG. 20 is a schematic illustration of a bone tap guide placed on a fifth metatarsal bone according to an aspect of the present disclosure.

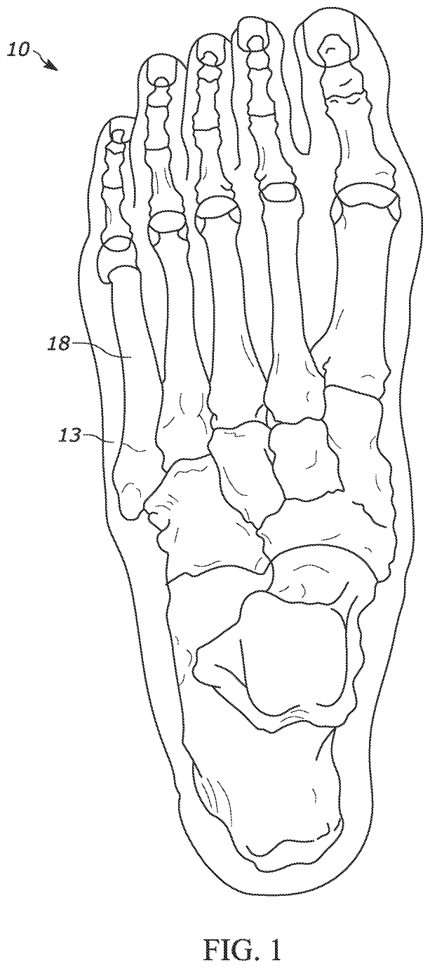

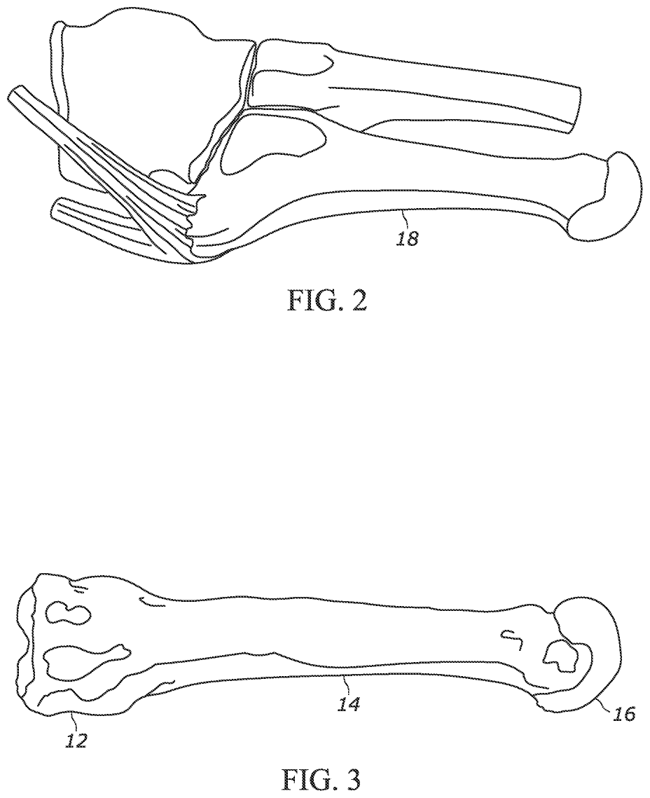

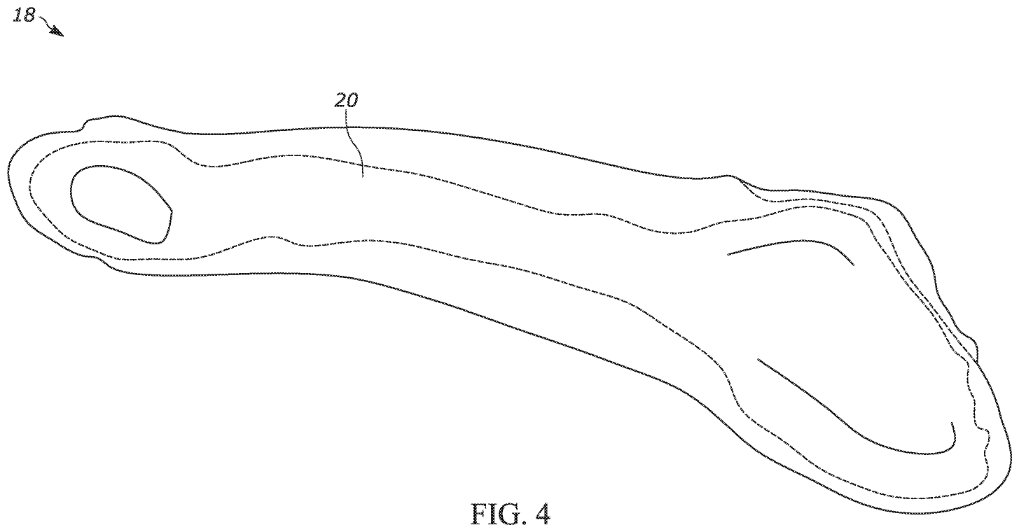

### DETAILED DESCRIPTION

As used herein with respect to a described element, the terms “a,” “an,” and “the” include at least one or more of the described element(s) including combinations thereof unless otherwise indicated. Further, the terms “or” and “and” refer to “and/or” and combinations thereof unless otherwise indicated. By “substantially” is meant that the shape or configuration of the referenced element need not have the mathematically exact described shape or configuration of the referenced element but can have a shape or configuration that is recognizable by one skilled in the art as generally or approximately having the described shape or configuration of the referenced element. As such “substantially” refers to the complete or nearly complete extent of a referenced characteristic. The exact allowable degree of deviation from the characteristic will be so as to have the same overall result as if the absolute characteristic were obtained. The terms “top,” “bottom,” “back,” and “front” refer to the position of elements as they are depicted in the drawings. The terms “first,” “second,” etc. are used to distinguish one element from another and not used in a quantitative sense unless indicated otherwise. Thus, a “first” element described below could also be termed a “second” element. By “integral” or “integrated” is meant that the referenced components are fabricated as one piece or multiple pieces affixed during manufacturing such that the components are a unitary and continuous piece after manufacturing. Such referenced components are not separable without damaging the integrity (i.e. tearing) of either of the components as opposed to two-piece or multi-piece components that are assembled together after manufacturing, such as prior to or during surgical insertion, and where the components can be separated. As used herein a “patient” includes a mammal such as a human being. All device, assemblies, and kits as described herein are used for medical purposes and are therefore sterile.

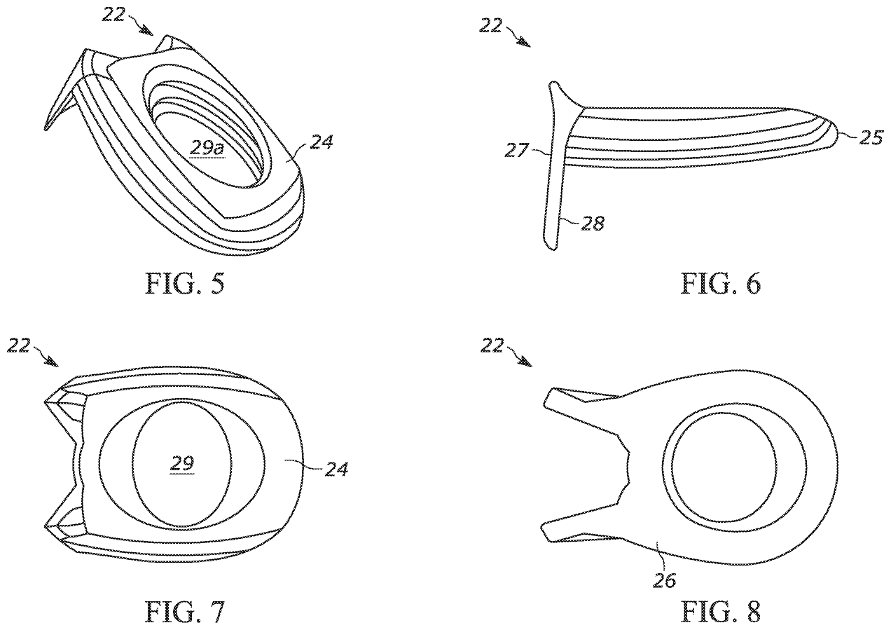

Referring to FIGS. 5-8, in an aspect, a bone fixation device 22 is provided. Device 22 comprises a front surface 25, a back surface 27 comprising at least one hook sized and configured to be placed at the base of the 5^{th} metatarsal bone of the foot, a top surface 24, a bottom surface 26, and an opening 29 extending between top surface 24 and bottom surface 26. Opening 29 is sized and configured to receive a bone screw. The bone fixation device is sized and configured to compress a proximal 5^{th} metatarsal fracture (a Jones fracture).

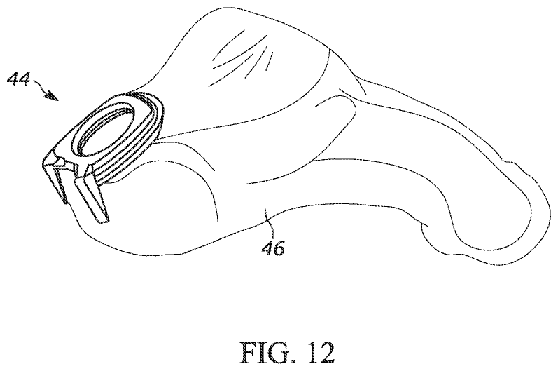

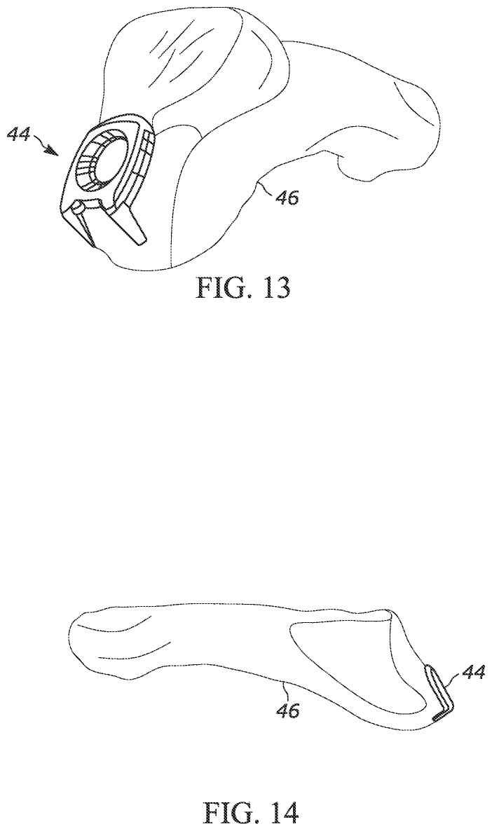

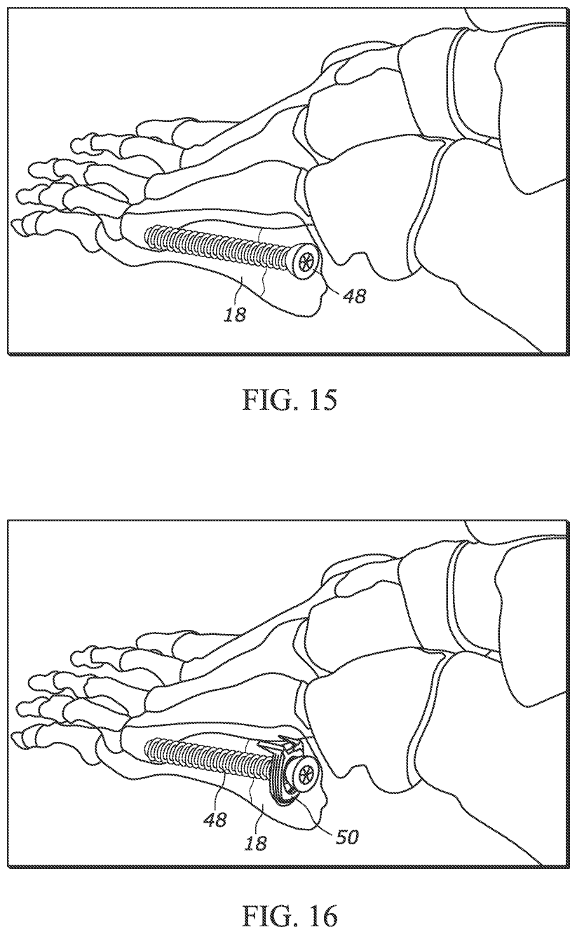

FIGS. 12-14 illustrate a fixation device 44 placed at the base of the 5^{th} metatarsal bone 46 of the foot. FIG. 15 schematically depicts a bone screw 48 inserted into the medullary canal of the 5^{th} metatarsal bone and FIG. 16 schematically illustrates fixation device 50 placed around the screwhead (although a headless screw could be used as well).

The fixation device allows a surgeon to have up to 15 degrees of freedom to place the screw. The entrance of the screw is determined according to the position of the medullary canal. In certain aspects, the top surface is flat in order to create maximum compression of the fracture. The fixation device is used with a screw during fracture fixation to optimize compression and minimize the risk of intrusion of the screwhead through cortical bone during screw insertion. Due to the large surface area of the fixation device, the load of the inserted screw is distributed and therefore a greater compressive force of the screw can be maintained. The hook(s) at the back surface of the device guide and maintain the correct position of the fixation device.

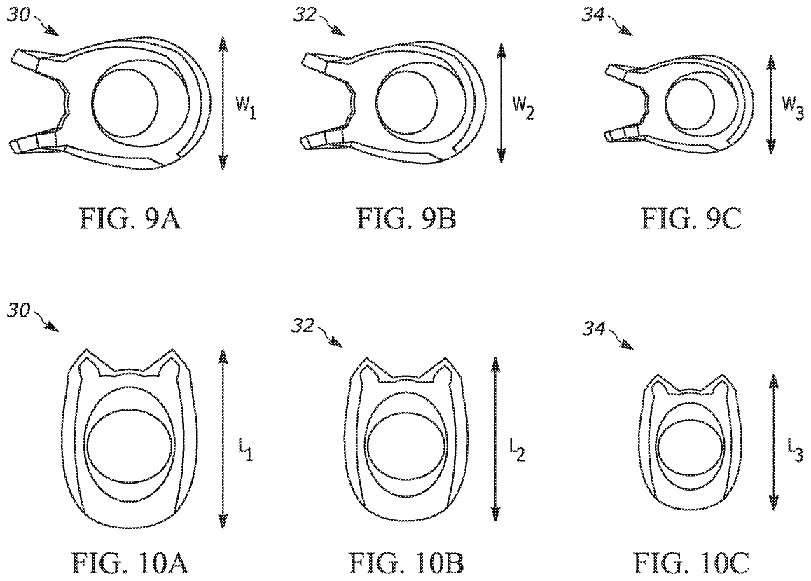

In certain aspects, a kit is provided with fixation devices 30, 32 and 34 having different widths (W) and lengths (L) as illustrated in FIGS. 9-10. The sizes of the fixation devices can depend on the size of the screw (e.g. 4.5 mm, 5.5 mm, 6.5 mm) that is being used. In certain aspects, the fixation device has a length of about 10.4 mm, a width of about 7 mm, a thickness of 2.0 mm. The hook(s) can be about 5.5 mm long.

As shown in FIG. 5, opening 29a can be threaded if the surgeon prefers to use a headless 5^{th} metatarsal screw. In such instance, the proximal part of the headless screw is locked by the threading into the fixation device.

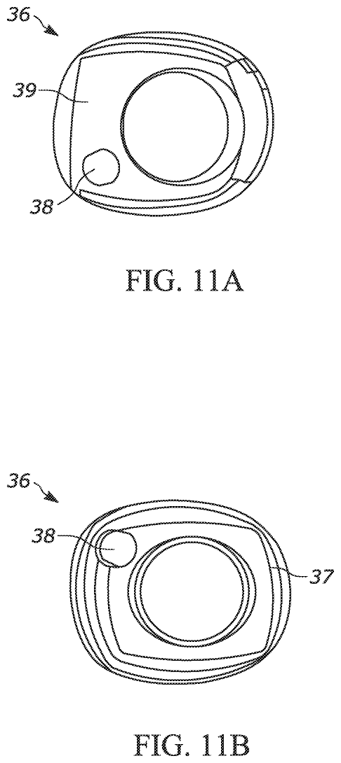

Referring to FIGS. 11A and 11B, a fixation device 36 can have different shapes. For example, top surface 37 and/or bottom surface 39 can have an oblong shape. The top and bottom surfaces can also define a small hole 38 extending therebetween to receive a K-wire. Through this hole, a K-wire can be placed to hold the fixation device in the desired position prior to screw placement.

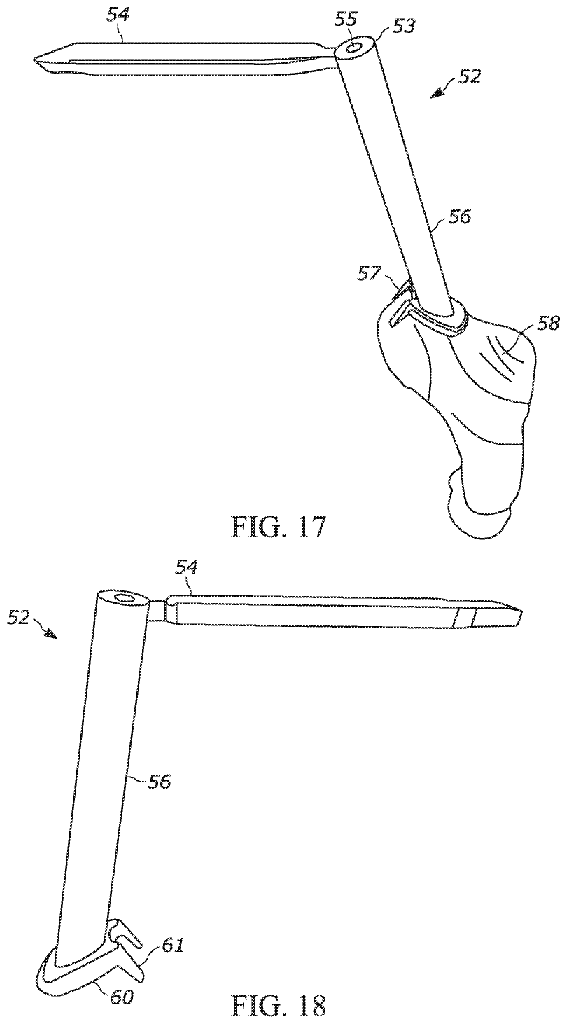

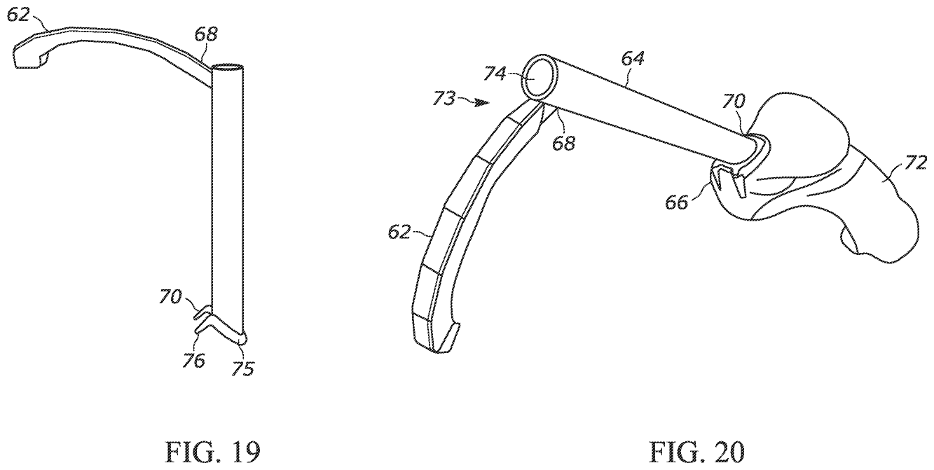

Referring to FIGS. 17-18, in an aspect, a drill guide 52 is provided. Drill guide 52 comprises a shaft 56 defining a bore 55 that is sized and configured to receive a K-wire. Drill guide 52 further includes a handle 54 extending proximally (as indicated by reference character 53 from shaft 56. The handle can be straight or curved as shown in FIG. 19 and as described in more detail below. A fixation device 60 as described above including at least one hook 61 extends distally (as indicated by reference character 57) from shaft 56 and is sized and configured to fit onto a 5^{th} metatarsal bone 58, such as the base on the bone.

Referring to FIG. 19-20, a bone tap guide 73 is also provided comprising a shaft 64 defining a bore 74 that is sized and configured to receive a bone tap. Bone tape guide 73 further includes a handle 62, which is depicted in FIG. 19 as being curved but can also be straight extending proximally (as indicated by reference character 68) from shaft 64. A fixation device 75 as described above including at least one hook 76 extends distally (as indicated by reference character 70) extends distally from shaft 64 and is sized and configured to fit onto a 5^{th} metatarsal bone 72, such as the base on the bone.

Each of the disclosed aspects and embodiments of the present disclosure may be considered individually or in combination with other aspects and embodiments. Further, while certain features of embodiments may be shown in only certain figures, such features can be incorporated into or deleted from other embodiments shown in other figures or otherwise disclosed in the specification. Additionally, when describing a range, all points within that range are included in this disclosure.

What is claimed is:

1. A bone fixation device comprising:
a front surface,
a back surface comprising at least one hook sized and configured to be placed at the base of the 5^{th} metatarsal bone of the foot;
a top surface;
a bottom surface; and
an opening extending between the top surface and the bottom surface, the opening sized and configured to receive a bone screw, wherein the bone fixation device is sized and configured to compress a proximal 5^{th} metatarsal bone fracture.

2. The device of claim 1, wherein the at least one hook comprises two hooks.

3. The device of claim 1, wherein the opening is a threaded opening.

4. The device of claim 1, wherein the device has a width of between about 7 and about 12 millimeters (mm) and a height of between about 10 and about 14.5 mm.

5. The device of claim 1, wherein the top surface is substantially flat.

6. The device of claim 1, wherein the top surface has an oblong shape.

7. The device of claim 1, wherein the bottom surface has an oblong shape.

8. The device of claim 1, wherein the top surface and the bottom surface define an additional opening sized and configured to receive a K-wire.

9. A bone fixation guide comprising:
a shaft defining a bore, the bore sized and configured to receive a K-wire;
a handle extending proximally from the shaft; and
at least one hook extending distally from the shaft and sized and configured to fit onto a 5^{th} metatarsal bone.

10. The bone fixation guide of claim 9, wherein the at least one hook comprises two hooks.

11. The bone fixation guide of claim 9, wherein the handle is curved.

12. The bone fixation guide wherein the bore has a diameter of between about 1.6 mm to about 2.0 mm.

13. The bone fixation guide of claim 9, wherein the bore has a diameter of between about 4.5 mm to about 6.5 mm.
